(19) United States
(12) Patent Application Publication
(10) Pub. No.: US 2005/0107878 A1
(43) Pub. Date: May 19, 2005

(54) Spinal intervertebral implant adjustable in situ

(75) Inventor: Frédéric Conchy, Saint-Médard d'Eyrans (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Stryker Spine, Cestas (FR)

(21) Appl. No.: 10/433,531

(22) PCT Filed: Dec. 5, 2001
(86) PCT No.: PCT/FR01/03837

(30) Foreign Application Priority Data
Dec. 5, 2000 (FR) 00/15737

(51) Int. Cl. A61F 2/44

(52) U.S. Cl. 623/17.11

(57) ABSTRACT

The invention concerns a spinal intervertebral implant (100) comprising at least a first element (101′) having a first end (112′), and a second element (101) having a second end (112), each end having successive ramps (108, 116), the ramps of the two ends being adapted to co-operate mutually to vary one dimension of the implant depending on the relative position of the elements. The invention is characterised in that the ramps of each end are arranged along a circle.

The invention concerns implants of the intervertebral cage type, or of the type for replacement of vertebral bodies, intended for the spinal column.

The document U.S. Pat. No. 5,865,848 discloses an intervertebral cage intended to replace a damaged intervertebral disk and comprising two parts which complement one another. These two complementary parts are able to move relative to one another in the direction of their greatest length. Height adjustment is made possible by the presence of contact surfaces, between the two parts of the cage, comprising ramps toothed in the direction of movement. The disadvantage of such a configuration is that it has a limit stop upon adjustment, necessitating a reverse movement in the event of an error. This reverse movement is awkward and difficult during the surgical intervention and risks causing instability between the vertebrae which are being operated on, said instability being prejudicial to achieving fusion between these operated vertebrae.

It is an object of the invention to permit height adjustment without reverse movement being necessary.

For this purpose, the invention provides a spinal intervertebral implant comprising at least a first element having a first end, and a second element having a second end, each end having successive ramps, the ramps of the two ends being able to cooperate mutually in order to vary one dimension of the implant depending on the relative position of the elements, and the ramps of each end being arranged along a circle.

Thus, the height of the implant is adjusted by rotating one of the elements on an axis passing through the center of the circle of ramps. The adjustment is continuous because it has no limit stop: it is possible to return to the initial position in the same sense of rotation, even in the event of an error.

Advantageously, some of the successive ramps are offset with respect to one another in the same sense in the direction of the dimension which is able to be varied.

Advantageously, the successive ramps of one end form groups of adjacent ramps comprising an identical number of ramps.

Thus, upon height adjustment, a constant minimum number of supports is ensured.

Advantageously, the groups are identical to one another.

Advantageously, the groups are uniformly distributed along the circle.

Advantageously, the circle comprises at least two groups of ramps.

Advantageously, the ends complement one another.

Advantageously, the implant comprises lateral orifices.

Advantageously, the implant comprises a central orifice extending along the dimension which is able to be varied.

Advantageously, the central orifice is able to receive substance promoting bone growth.

Advantageously, the implant comprises stabilizing means which are able to hold the elements relative to one another with respect to a direction of relative movement.

Thus, the stability of the implant is optimal.

Advantageously, the stabilizing means comprise a member which can be received in the central orifice.

Advantageously, the stabilizing means comprise at least one supporting element integral with at least one of the ends.

Advantageously, with one of the two elements, preferably the first one, having a third end with ramps, the implant comprises at least a third element having a fourth end with ramps able to cooperate with the ramps of the third end in order to vary the dimension of the implant depending on the relative position of the first and third elements.

Advantageously, the orientation of the ramps of the first end is mirror-symmetrical to that of the ramps of the third end, in a plane perpendicular to the direction of the dimension which is able to be varied. Thus, height adjustment requires only the movement of the intermediate element situated between the two others which thus remain immobile relative to the vertebrae operated on. This ensures better anchoring of the implant in the vertebrae.

Advantageously, the implant comprises terminal ends having teeth which are profiled and parallel to one another.

Advantageously, the implant comprises terminal ends having a face and points protruding from the face.

Provision is also made, according to the invention, for a surgical method which comprises the steps of fitting the implant at the implantation site and adjusting a dimension of the implant in situ by modifying a relative position of at least one of the elements of the implant.

Advantageously, the surgical method additionally comprises a step of filling the implant with a substance promoting bone growth.

Other characteristics and advantages of the invention will become evident from the following description of three preferred embodiments of the invention which are given as nonlimiting examples. In the attached drawings:

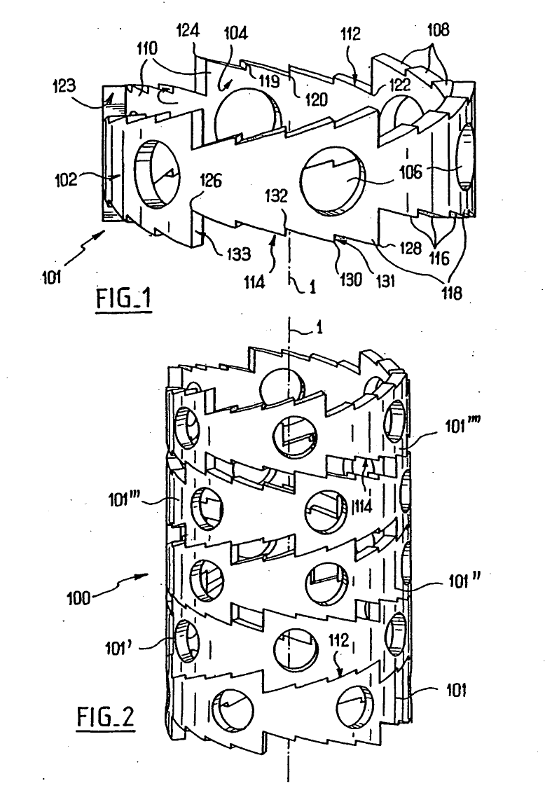

FIG. 1 is a view, in three dimensions, of a basic element in a first embodiment of the invention;

FIG. 2 is a view, in three dimensions, of the stacking of a certain number of basic elements from FIG. 1 in order to form an implant according to the first embodiment of the invention;

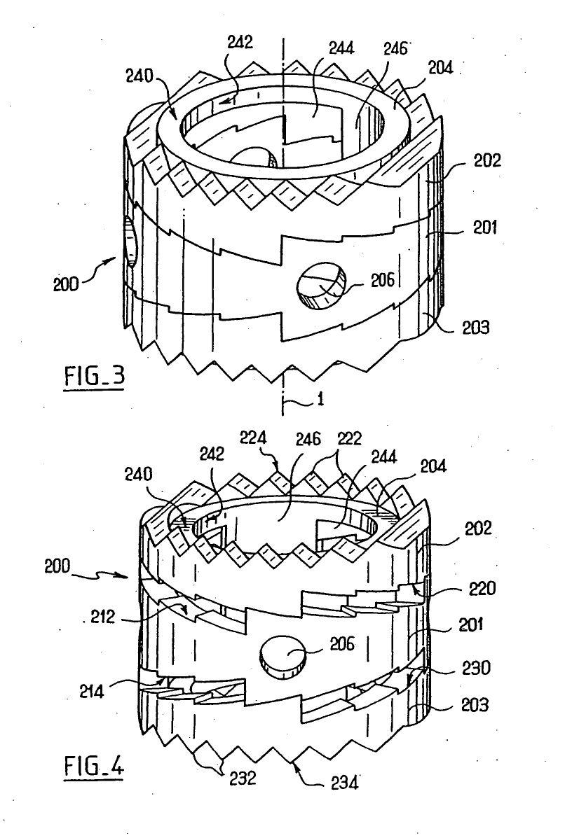

FIG. 3 is a view, in three dimensions, of a second embodiment of the invention in the position of minimum height;

FIG. 4 is a view, in three dimensions, of the second embodiment in the position of maximum height;

FIG. 5 is a view, in three dimensions, of a third embodiment of the invention; and

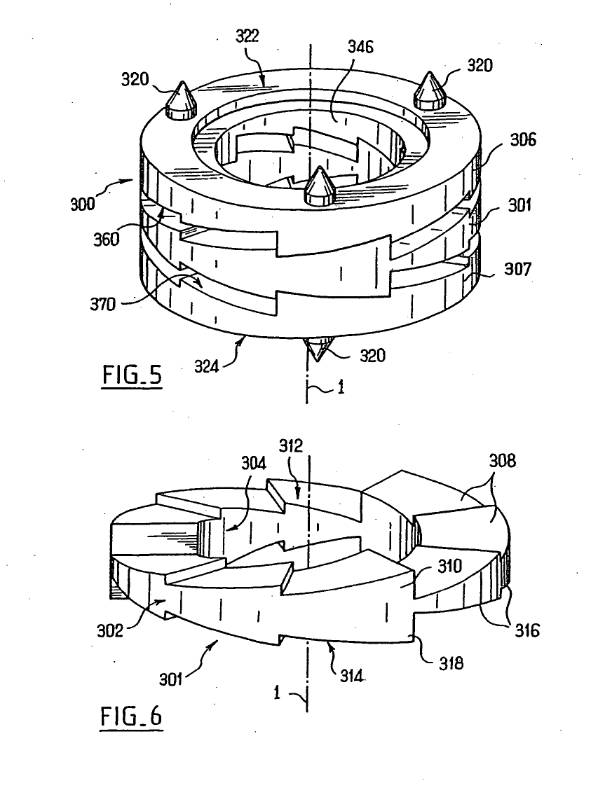

FIG. 6 is a view, in three dimensions, of the intermediate member of the third embodiment from FIG. 5.

The first embodiment of the invention will be described with reference to FIGS. 1 and 2. This first embodiment comprises a basic element 101 which, when stacked together with other identical basic elements 101, will constitute an implant 100 of the intervertebral cage type which is adjustable in situ. The basic element 101 is in the form of a ring with an outer face 102 and an inner face 104, both of them cylindrical. The inner face 104 delimits a central orifice. The basic element 101 additionally comprises a first contact surface or end 112 and a second contact surface or end 114, the two surfaces 112 and 114 being mirror-symmetrical in a median transverse plane perpendicular to an axis 1 of revolution of the ring forming the basic element 101.

Each of the contact surfaces 112 and 114 comprises a plurality of ramps 108 and 116, respectively, identical to each other. The ramps 108 are organized in groups 110 of adjacent ramps, here numbering four per group 110. All the groups 110 are identical to one another and are uniformly distributed along the upper surface 112. The ramps have a top point 119 and a bottom point 120, these two points forming the ends of each of the ramps 108. The ramps 108 within a group 110 are arranged in such a way that the top point 119 of one ramp is situated above the top point of the preceding ramp, but below that of the following ramp, the succession of ramps within a group 110 being in the opposite direction to the hands of a clock. The top point 119 of one ramp is connected to the bottom point 120 of the following ramp by a vertical wall 121. The bottom point 120 of the first ramp of the group 110 forms the bottom point 122 of the group 110, while the top point 120 of the last ramp of the group 110 forms the top point 124 of the group 110. The top point 124 of one group 110 is connected to the bottom point 122 of the following group by a vertical wall 123.

Similarly, but in a manner which is mirror-symmetrical in relation to a transverse plane perpendicular to the axis 1 of the ring 1, the ramps 116 are organized in groups 118 of adjacent ramps, these groups being uniformly distributed along the lower surface 114. The ramps 116 have a top point 132 and a bottom point 130 and are connected to one another by a vertical wall 131 within a group. Moreover, the bottom point of one group 118 is connected to the top point 126 of the following group by a vertical wall 133.

In an optimal manner, each group 118 on the lower face is positioned substantially in line with a group 110 on the upper face: thus, the top point 124 is vertically in line with the bottom point 128, thereby defining the maximum height of the basic element 101; likewise, the bottom point 122 is situated vertically in line with the top point 126, thereby defining the minimum height of the basic element 101. The arrangement of the various groups 110 along the surface 112 and that of the various groups 118 along the surface 114 are such that the ramps 108 and 116 respectively describe a circular trajectory whose axis coincides with that 1 of the ring forming the basic element 101.

Moreover, the outer face 102 comprises a plurality of through-orifices 106: these orifices open out, on the inner face 104, into the central orifice delimited by this face 104.

Having described the basic element 101, we will now discuss its use. Once the surgeon has accessed the implantation site and then prepared said implantation site, he forms an implant 100 by stacking together a plurality of basic elements 101. Here, by way of illustration, there are five basic elements 101, 101′, 101″, 101′″, 101″″. Two elements are respectively stacked on one another by turning the second element around relative to the first element in order to bring the contact surface 112, 114 of the first basic element into contact with its counterpart 112, 114, respectively, of the second basic element. Thus, the basic element 101′ is turned round so that its upper surface 112 comes into contact with the upper surface 112 of the basic element 101. In this way, the two surfaces complement one another. Then, the basic element 101″ is stacked on the basic element 101′ in such a way that the lower surface 114 of the basic element 101″ comes into contact with the lower surface 114 of the basic element 101′. This procedure is continued to stack the subsequent basic elements.

Once the implant 100 has been formed, the surgeon fills the central orifice delimited by the inner face 104 of each of the basic elements 101 with an osteoinductive or osteoconductive substance such as a bone graft (allograft or autograft), hydroxyapatite, or tricalcium phosphate (TCP), etc.

The surgeon then places the implant 100 in the implantation site. He impacts the ramps of the lower face of the basic element 101 into the upper plateau of the lower vertebra of the site. Likewise, he impacts the ramps of the upper face of the basic element 101″″ into the lower plateau of the upper vertebra of the site. The ramps thus serve in both cases as anchoring means for the implant 100. The surgeon is then able to adjust the height of the implant 100 in situ by rotating one or more of the various intermediate basic elements 101′, 101″, 101′″. He thus obtains the desired height between the two vertebrae, i.e. the upper vertebra and lower vertebra, which delimit the implantation site. The final step involves closing the access route.

A second embodiment of the invention will now be described with reference to FIGS. 3 and 4. The implant 200 of the intervertebral cage type according to this second embodiment comprises an intermediate element 201, an upper plate 202, a lower plate 203, and stabilizing means 204.

The intermediate element 201 is in the form of a ring and is quite similar to the basic element 101 in the previous embodiment. It comprises an upper surface 212 with uniformly distributed groups of ramps, describing a circular trajectory and similar to the groups 110 in the previous embodiment. Likewise, it comprises a lower surface 214 with uniformly distributed groups of ramps, describing a circular trajectory and similar to the groups 118 in the previous embodiment. Like the basic element 101, the intermediate element 201 comprises lateral orifices 206 passing through the thickness of the ring which forms said intermediate element and opening into the central orifice.

The upper plate 202 is in the form of a ring having the same internal and external diameters as those of the intermediate element 201. It comprises a lower surface 220 which is able to come into contact with the upper surface 212 of the intermediate element 201. This surface 220 is complementary to the surface 212. The plate 202 additionally comprises an upper face 224 with anchoring means 222 for anchoring to bone material such as vertebral plateaus. These anchoring means 222 are in this case teeth which are of triangular profile with slopes at 45° with respect to the horizontal and perpendicular to one another. The teeth are, furthermore, parallel to one another.

The lower plate 203 is the symmetrical counterpart of the upper plate 202, these being mirror-symmetrical in a transverse plane perpendicular to the axis 1 of the ring. It thus comprises an upper surface 230 which is able to come into contact with the lower surface 214 of the intermediate element 201, with which surface it is complementary. The plate 203 additionally comprises a lower face 234 having means 232 for anchoring to bone material which are identical to the anchoring means 222 of the upper plate 202.

The stabilizing means 204 comprise a component of revolution whose external diameter is substantially equal to the internal diameter of the ring forming the plates 202, 203 and the intermediate element 201. Thus, the stabilizing means 204 are able to be received with sliding along the axis of revolution in the central orifice of the implant 200. The component of revolution is in this case a tube comprising two end faces 240, namely upper face and lower face, and an inner face 242 delimiting a through-hole 246. The wall of the tube is openworked with openings 244. Thus, the lateral orifices 206 can still communicate with the inside of the implant 200 as represented by the hole 246. The stabilizing means 204 have the role of preventing any sliding, in a substantially radial direction, of one of the plates 202, 203 relative to the intermediate element 201.

The positioning of such an implant 200, in a surgical intervention, is similar to that described for the previous embodiment. After accessing and preparing the implantation site, the surgeon forms an implant 200 which he positions at its minimum height as illustrated in FIG. 3: the surface 220 is totally in contact with the surface 212, and the surface 230 is totally in contact with the surface 214. The end faces 240 are therefore flush with the summits of the profiled teeth 222 and 234, respectively. The implant is said to be in its lowered configuration. The hole 246 is then filled with an osteoinductive or osteoconductive substance. The implant is then placed in the implantation site. The surgeon impacts the toothed faces of the plates 202, 203 into the vertebral plateaus delimiting the implantation site from above and below. then he sets the desired height by maneuvering the intermediate element 201 in rotation.

It is of course possible to obtain greater heights by stacking several intermediate elements 201 between the two plates 202 and 203. This stacking is done identically to that of the basic elements 101 in the previous embodiment.

Referring to FIGS. 5 and 6, a third embodiment of the invention will now be discussed. the implant 300 of the intervertebral cage type in this third embodiment is almost identical to the previous embodiment in that it has an intermediate element 301 and two plates, namely an upper plate 306 and a lower plate 307.

The intermediate element 301 is in the form of a ring and has a lower surface 314 and an upper surface 312. The upper surface 312 comprises a plurality of groups 310 of ramps 308 uniformly distributed on the surface 312 and identical to one another and arranged in the same way as the groups 110 and 210 in the previous embodiments. Likewise; the lower surface 314 comprises a plurality of groups 318 of ramps 316 uniformly distributed on the whole surface 314 and identical to one another and arranged in the same way as the groups 118 and 218 in the previous embodiments. The intermediate element 301 additionally comprises an outer face 302 and an inner face 304 delimiting a continuous central orifice. The width of the ring, that is to say the distance between the inner face 304 and outer face 302 forming the intermediate element, is greater than that of the rings forming the basic element 101 and intermediate element 201. Thus, the lower and upper surfaces are larger and make it possible to obtain a much more stable support, as will be seen below.

The upper plate 306 is in the form of a ring with internal and external diameters almost identical to those of the ring forming the intermediate element 301. The plate 306 has a lower surface 360 able to come into contact with the upper surface 312 of the intermediate element 301. The lower surface 360 of the plate 306 is complementary to the upper surface 312 of the intermediate element 301. Moreover, the upper plate 306 has an upper face 322 which is plane and perpendicular to the axis 1 of the ring forming the plate. However, in an alternative embodiment, this face 322 can be inclined relative to a plane perpendicular to the axis of the ring. The plate 306 additionally comprises anchoring means 320 which in this case are points protruding from the face 322 and of circular cross section. The points 320 protrude perpendicularly to the face 322.

The lower plate 307 is the mirror-symmetrical counterpart of the upper plate 306 with respect to a transverse plane perpendicular to the axis 1 of the ring. It thus has an upper surface 370 symmetrical to the surface 360 and able to come into contact with the lower surface 314 of the intermediate element 301 in a complementary fashion. The lower plate 307 has a lower face 324 symmetrical to the face 322.

The use and positioning of an implant 300, during a surgical intervention, is similar to the use and positioning of the implant 200 in the previous embodiment. It should be noted that, upon impaction into the upper and lower vertebral plateaus delimiting the implantation site, it is the points 320 which are driven into the bone of said vertebral plateaus.

It is of course possible for numerous modifications to be made to the present invention without departing from the scope of the latter.

The faces comprising the anchoring means can be inclined with respect to a plane perpendicular to the main axis of the implant.

The stabilizing means can comprise a supporting element formed integrally on at least one of the contact surfaces of each pair and protruding in the direction of bearing on one of the faces, inner or outer, of the elements constituting the implant.

An implant can be provided which comprises three elements, i.e. an intermediate element whose ends are able to come into contact with one of the ends of each of the other two elements.

It is possible to conceive of any cam system with cam follower other than those described above, without departing from the present invention.

1. A spinal intervertebral implant (100; 200; 300) comprising at least a first element (101′; 201; 301) having a first end (112; 214; 314), and a second element (101; 203; 307) having a second end (112; 230; 370), each end having successive ramps (108, 116; 308, 316), the ramps of the two ends being able to cooperate mutually in order to vary one dimension of the implant depending on the relative position of the elements, characterized in that the ramps of each end are arranged along a circle.

2. The implant as claimed in claim 1, characterized in that some of the successive ramps are offset with respect to one another in the same sense in the direction of the dimension which is able to be varied.

3. The implant as claimed in claim 1 or 2, characterized in that the successive ramps of one end form groups (110, 118; 310, 318) of adjacent ramps comprising an identical number of ramps.

4. The implant as claimed in claim 3, characterized in that the groups are identical to one another.

5. The implant as claimed in claim 3 or 4, characterized in that the groups are uniformly distributed along the circle.

6. The implant as claimed in one of claims 3 through 5, characterized in that the circle comprises at least two groups of ramps.

7. The implant as claimed in one of the preceding claims, characterized in that the ends complement one another.

8. The implant as claimed in one of the preceding claims, characterized in that each element comprises lateral orifices (106; 206).

9. The implant as claimed in one of the preceding claims, characterized in that it comprises a central orifice extending along the dimension which is able to be varied.

10. The implant as claimed in one of the preceding claims, characterized in that it comprises stabilizing means (204) which are able to hold the elements relative to one another with respect to a direction of relative movement.

11. The implant as claimed in claim 10, characterized in that the stabilizing means comprise a member (204) which can be received in the central orifice.

12. The implant as claimed in claim 10, characterized in that the stabilizing means comprise at least one supporting element integral with at least one of the ends.

13. The implant as claimed in one of the preceding claims, characterized in that, with one of the two elements (101′; 201; 301), preferably the first one; having a third end (114; 212; 312) with ramps (116; 308), the implant comprises at least a third element (101″; 202; 306) having a fourth end (114; 220; 360) with ramps able to cooperate with the ramps of the third end in order to vary the dimension of the implant depending on the relative position of the first and third elements.

14. The implant as claimed in claim 13, characterized in that the orientation of the ramps of the first end is mirror-symmetrical to that of the ramps of the third end, in a plane perpendicular to the direction of the dimension which is able to be varied.

15. The implant as claimed in one of the preceding claims, characterized in that it comprises terminal ends (224, 234) having teeth (222, 232) profiled and parallel to one another.

16. The implant as claimed in one of claims 1 through 14, characterized in that it comprises terminal ends (322, 324) having a face (322, 324) and points (320) protruding from the face.
